(19) United States
(12) Patent Application Publication
(10) Pub. No.: US 2004/0230269 A1
(43) Pub. Date: Nov. 18, 2004

(54) Medical lead adaptor assembly

(75) Inventors: Eric W. Huff, St. Paul, MN (US); Frank L. Skubitz, Andover, MN (US); Timothy W. Holleman, Ham Lake, MN (US); Timothy C. Aarons, Maple Grove, MN (US); Mark Tobritzhofer, Fridley, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/436,960

(22) Filed: May 13, 2003

(51) Int. Cl. A61N 1/00

(52) U.S. Cl. 607/116

(57) ABSTRACT

An adaptor facilitating electrical connection between an implantable medical lead connector and an external medical device includes a longitudinally extending connector receptacle formed by a first and second housing portion flexibly joined together. An inner surface of the connector receptacle is opened, to facilitate insertion of the connector therein, by means of a first lever arm extending laterally from the first housing portion and a second lever arm extending laterally from the second housing portion. An electrical contact formed within the inner surface of the connector receptacle is adapted to electrically engage at least one ring contact of the connector when the connector is fully inserted within the connector receptacle. An external contact surface, formed upon the first lever arm and electrically coupled to the electrical contact via a conductive pathway, is adapted for coupling with a contact element of the external medical device.

### CROSS REFERENCE TO RELATED APPLICATION

Cross-reference is hereby made to commonly assigned related U.S. Applications filed concurrently herewith: No. XX/XXX,XXX to William Wenger, entitled “Medical Lead Adaptor Assembly” (Attorney Docket No. P-11376.00) and No. XX/XXX,XXX to Timothy Holleman et al., entitled “Medical Lead Adaptor Assembly” (Attorney Docket No. P-11106.00).

### FIELD OF THE INVENTION

The present invention generally relates to a medical lead adaptor assembly, and in particular, the present invention relates to a medical lead adaptor assembly facilitating a temporary connection between a medical lead of an implantable medical device and an external medical device.

### BACKGROUND OF THE INVENTION

The earliest instances of relatively prolonged cardiac stimulation, namely cardiac pacing, of a patient's heart was effected through implanted cardiac leads attached to the heart muscle at distal electrode ends and extending through an incision in the patient's skin. To effect unipolar pacing of the heart, a single such implantable pacing lead was employed in conjunction with a subcutaneously implanted or skin-surface attached return electrode coupled to an external lead conductor. To effect bipolar pacing of the heart, two such implantable pacing leads were implanted with the electrode ends implanted a distance apart. The attachment of the proximal ends of the lead conductors to the temporary cardiac pacemaker connector elements was initially effected by simply stripping insulation from the proximal conductor ends, and inserting and securing the bare conductor ends in transverse openings in threaded posts. Later, finished connector pins were formed at the proximal connector ends of the lead bodies that could be inserted into the end openings of thumb nuts and connector posts.

Implantable pacing leads evolved into permanent, unipolar and bipolar, endocardial and epicardial, pacing leads for chronic implantation in a patient. The proximal electrical connector assemblies were then connected with connector elements of a totally implanted, cardiac pacemaker pulse generator. To withstand stress, implantable pacing lead conductors were formed of coiled wire and inserted within an insulative lead body lumen, thereby providing a coiled wire lumen that was sized to receive a stiffening stylet wire to assist transvenous implantation of the endocardial pacing leads. The proximal end of the coiled wire conductor was attached to a tubular connector pin at the terminus of the lead connector and shaped to be received in the connector assembly of the implantable pacemaker pulse generator. In the case of endocardial permanent pacing leads, the connector or pin was formed with a lumen therein aligned with the coiled wire lumen so that the stiffening stylet wire could be inserted down the length of the lead body during the transvenous introduction and withdrawn after placement of the distal electrode was achieved. Many of these features are employed in current permanent pacing leads.

More recently, bipolar and multi-polar permanently implantable pacing leads and leads for use in pacing and cardioversion/defibrillation (collectively referred to as permanent implantable cardiac leads) have been developed using coaxially arranged, coiled wire conductors and/or parallel-wound, multi-filar coiled wire conductors. In the case of endocardial cardiac leads, the stylet wire lumen is employed to receive the stiffening stylet wire for implantation as described above. The proximal connector end assemblies are formed with at least two spaced apart lead connector elements arranged in-line from a proximal lead connector pin to at least one or more distally located ring-shaped element or lead connector ring. Typical bipolar in-line lead connector assemblies for multi-filar, coiled wire conductors are shown, for example, in commonly assigned U.S. Pat. Nos. 4,944,088 and 4,951,687 and 5,007,435, respectively, the teachings of which are hereby incorporated by reference.

Unipolar and bipolar, temporary endocardial pacing leads and temporary epicardial heart wires were also developed for implantation of the distal electrode(s) thereof in contact with the endocardium or sutured through the epicardium of the hearts of hospitalized patients. The lead body size of these temporary pacing leads and heart wires has typically been smaller than that of permanent cardiac leads because of the absence of an internal wire coil lumen for receiving a stylet wire. Still, in the case of bipolar temporary pacing leads and heart wires, either a lead connector pin and ring set are employed providing a pair of lead connector pins.

During or after implantation of the implantable cardiac lead(s), an external analyzer, e.g. a MEDTRONIC® Model No. 's 2290 and 8090, is attached to the proximal lead connector end assembly accessible through the incision to assess the performance of the system and verify proper lead placement. It is necessary in some cases to use either a disposable or a reusable “surgical cable” adaptor to complete the connection between the implanted lead and the external pacing system analyzer.

Some patient and surgical cable adaptors constitute a connector assembly at a first end that is compatible with the analyzer or temporary pacemaker terminals, a cable including conductors extending from the first end to a second end, and lead connector element connectors at the second end. Typically, two to four conductors are included in the cable, and a set of two or four alligator clips are provided at the second end for attachment to one or more lead connector rings and a pin of one or two implantable cardiac leads.

In the case of a permanent pacing lead having a stylet wire fitted within the lead lumen and projecting out proximally through the connector pin, alligator clips are utilized that attach across the connector rings and pins.

However, such an attachment is not as secure and electrically isolated as would be desirable. It is undesirable to either lose the connection or to allow an electrical static discharge or other shock or impulse to reach the heart through the exposed lead connector ends. Furthermore, it has been observed that the careless use of alligator clips can damage the insulation sheathes adjacent to the lead connector end ring or connector pins. This problem is further complicated in the case of leads having a plurality of contact rings separated by insulative sealing surfaces. That is, not only is there a potential for shorting between alligator clips and/or test probes, but such clips may cause damage to the insulation/sealing areas adjacent the contact rings.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a simplified schematic view of a cardiac lead implanted in a patient and coupled to an external medical device by means of an inventive medical lead adaptor;

FIG. 2 is an isometric view of a lead connector assembly capable of being received into an inventive medical lead adaptor;

FIGS. 3A and 3B are an end view and a side plan view, respectively, of an adaptor according to one embodiment of the present invention;

FIG. 3C is an end view of an alternate embodiment of an adaptor according to the present invention;

FIG. 4A is an isometric view of an adaptor according to yet another embodiment of the present invention.

FIG. 4B is an isometric view of a conductive assembly included in the adaptor illustrated in FIG. 4A.

FIG. 5 is an isometric view of a lead connector engaged within the adaptor illustrated in FIG. 4A.

FIG. 6A is an isometric view of an adaptor according to yet another embodiment of the present invention; and

FIGS. 6B and 6C are side and end views, respectively, of the adaptor illustrated in FIG. 6A.

### DETAILED DESCRIPTION OF THE INVENTION

The following detailed description of the invention is merely exemplary in nature and is not intended to limit the scope, applicability, or configuration of the invention in any way. Rather, the following description provides a convenient illustration for implementing exemplary embodiments of the invention. Various changes to the described embodiments may be made in the function and arrangement of the elements described herein without departing from the scope of the invention.

The invention is described in connection with a number of embodiments of medical lead adaptor assemblies, each of which allows electrical coupling between the proximal lead connector end assembly of a cardiac or similar lead and an external medical device. The lead adaptor is capable of being coupled to external electrical conductors by means of conductive probes, clips, and the like. The inventive medical lead adaptor assembly may be configured to accept lead connectors that may or may not include a stylet wire or a guide wire passing therethrough. Furthermore, the inventive lead adaptor may be utilized in conjunction with leads having compatible lead connector element dimensions; i.e. compatible spacing between and diameters of ring contacts. Of course, the medical lead adaptor assembly in accordance with the present invention may be provided with different dimensions so as to accommodate a variety of cardiac or other types of leads.

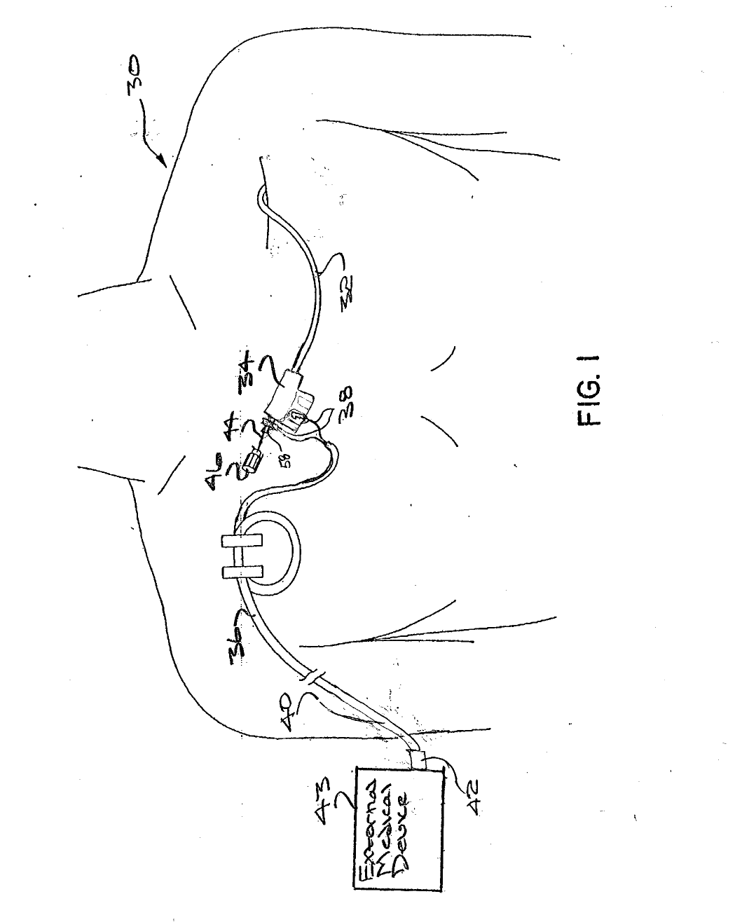

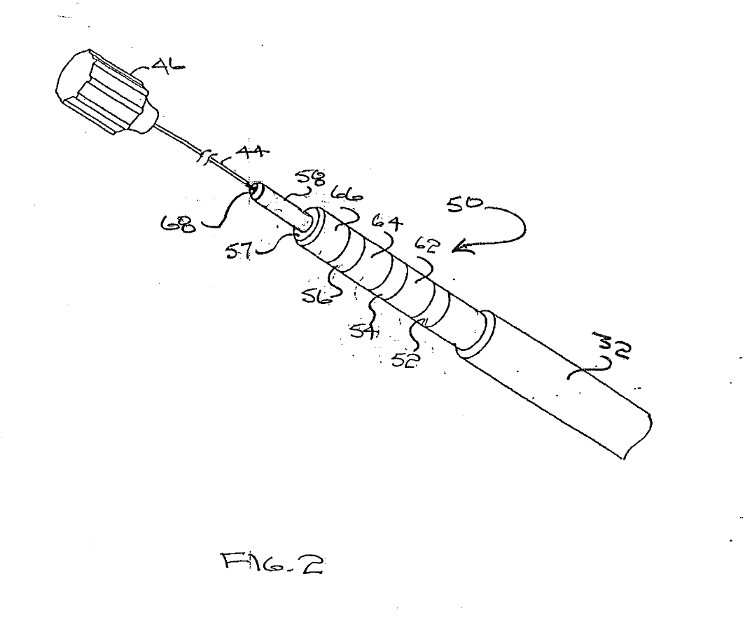

FIG. 1 is a simplified schematic view of a cardiac lead implanted in a patient and coupled to an external medical device by means of an inventive medical lead adaptor. As can be seen, a proximal portion of an implantable cardiac lead is shown in part and includes an elongated implantable lead body 32 extending from a lead adaptor assembly 34 toward the distal cardiac lead end (not shown). The distal cardiac lead end includes at least one electrode implanted in contact with a heart chamber of patient 30. The lead connector (shown in FIG. 2 as 50) is received within adaptor 34 as will be described hereinafter for providing rapid electrical connection between lead body 32 and external medical device 43 by means of a cable 36 and one or more contact elements, examples of which include alligator clips 38, as illustrated in FIG. 1, and probes. The proximal end 40 of cable 36 is provided with means for electrical connection to one or more external medical devices by means of, for example, a connector 42 that engages a connector terminal associated with the external medical device. The external medical device connection terminal may take any form, such as those associated with the above-referenced MEDTRONIC® Models 2290 and 8090 or Model 5348 and 5388 temporary pacemakers. A stylet wire 44 extends through connector assembly 50 and lead body 32; alternately an interventional guide wire may extend through connector assembly 50 and lead body 32. In this manner, stylet wire 44, or a guide wire, may be rotated, axially extended, withdrawn, etc., to aid in implantation of lead body 32.

FIG. 2 is an isometric view of a lead connector assembly capable of being received into an adaptor according to the present invention, various embodiments of which are described herein. Connector 50 at the proximal end of lead body 32 includes contact rings 52, 54 and 56 and a pin contact 58, each electrically coupled to conductors within lead body 32 and electrically isolated from each other by insulative layers within lead body 32 and by sealing rings 62, 64, and 66. Extending from a lumen 68 in lead connector 50 is stylet wire 44 which may be manipulated by means of stylet knob 46 as described above. While connector 50 has been shown as comprising three contact rings and three insulative sealing rings, it should be clear that the inventive medical lead adaptor assembly is equally applicable to connectors having a different number of contact rings including a single contact ring as is typical of IS-1 connectors.

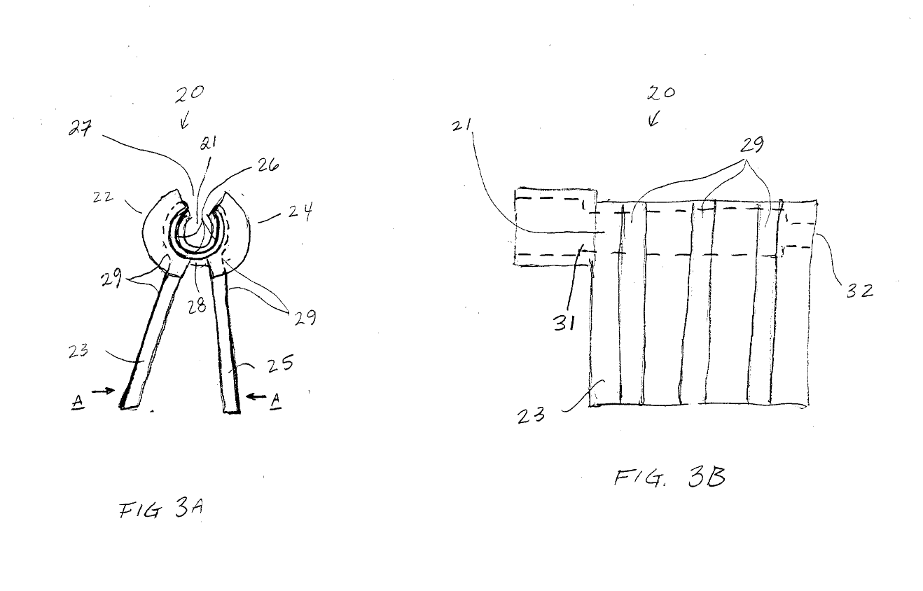

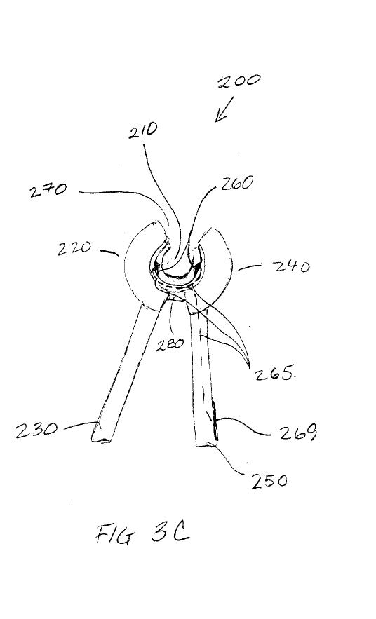

FIGS. 3A and 3B are an end view and a side plan view, respectively, of an adaptor 20 according to one embodiment of the present invention. FIGS. 3A and 3B illustrate adaptor 20 including a first housing portion 22 and a second housing portion 24 flexibly joined along a base 28 to form a connector receptacle 21 extending longitudinally from a proximal end 32 to a distal end 31 and including a slot 27 likewise extending; electrical contacts 26 are formed within connector receptacle 21, generally conforming to an inner surface of connector receptacle, and adapted to electrically engage contact rings of a connector, for example contact rings 52, 54 and 56 of connector 50 illustrated in FIG. 2. A first lever arm 23 and a second lever arm 25 are illustrated extending laterally from first housing portion 22 and second housing portion 24; according to embodiments of the present invention lever arms 23, 25 are adapted to spread housing portion 22, 24 apart when lever arms 23, 25 are forced toward one another per arrows A. According to embodiments of the present invention housing portions 22, 24, and lever arms 23, 25 are formed from a relatively hard plastic, preferably having physical properties to form a living hinge at base 28, examples of which include but are not limited to polysulfone, polypropylene and styrene-butadiene. FIGS. 3A and 3B further illustrate electrical contacts 26 extending out from connector receptacle 21 via slot 27 to form external contact surfaces 29 along housing portions 22, 24 and lever arms 23 and 25; according to embodiments of the present invention external contact surfaces 29 allow electrical coupling of a connector inserted within adaptor 20 to an external medical device, via contact elements such as alligator clips 38 illustrated in FIG. 1. Electrical contacts 26 and external contact surfaces 29 may be formed of strips or wires made of any material having an appropriate electrical conductivity and resiliency to allow opening of connector receptacle 21 when lever arms 23, 25 are forced toward one another, examples of which include stainless steel, titanium and platinum-iridium alloys. According to embodiments of the present invention, inner surface of connector receptacle 21 wherein electrical contacts 26 are formed, is dimensioned to tightly engage a lead connector, for example connector 50 illustrated in FIG. 2, in order to provide stable mechanical and electrical coupling, therefore lever arms 23, 25 must be pushed together (arrows A) in order to open up connector receptacle to allow insertion of a lead connector with minimal to no insertion force required. Although FIG. 3A illustrates electrical contact 26 extending to external contact surfaces 29 on both lever arms 23, 25, alternate embodiments according to the present invention include an external contact surface formed only along one of arms 23, 25.

FIG. 3C is an end view of an adaptor 200 according to an alternate embodiment. FIG. 3C illustrates adaptor 200 including a first housing portion 220 and a second housing portion 240 flexibly joined along a base 280 to form a connector receptacle 210. Connector receptacle 210 extends longitudinally from a proximal end to a distal end, as shown in FIG. 3B as 32 and 31, respectively and includes a slot 270 likewise extending. A first lever arm 230 and a second lever arm 250 are illustrated extending laterally from first housing portion 220 and second housing portion 240; according to embodiments of the present invention lever arms 230, 250 are adapted to spread housing portion 220, 240 apart when lever arms 230, 250 are forced toward one another as was described in conjunction with FIG. 3A. According to embodiments of the present invention housing portions 220, 240, and lever arms 230, 250 are formed from a relatively hard plastic, preferably having physical properties to form a living hinge at base 280, examples of which include but are not limited to polysulfone, polypropylene and styrene-butadiene. FIG. 3C further illustrates connector receptacle 210 including electrical contacts 260 formed as discrete protrusions from an inner surface of receptacle 210; electrical contacts are adapted to electrically engage contact rings of a connector, for example contact rings 52, 54 and 56 of connector 50 illustrated in FIG. 2, and are joined via internal conductive pathways 265, shown by dashed lines, to an external contact surface 269 formed on second lever arm 250. According to embodiments of the present invention external contact surface 269 allows electrical coupling of a connector inserted within adaptor 200 to an external medical device, via contact elements such as alligator clips 38 illustrated in FIG. 1.

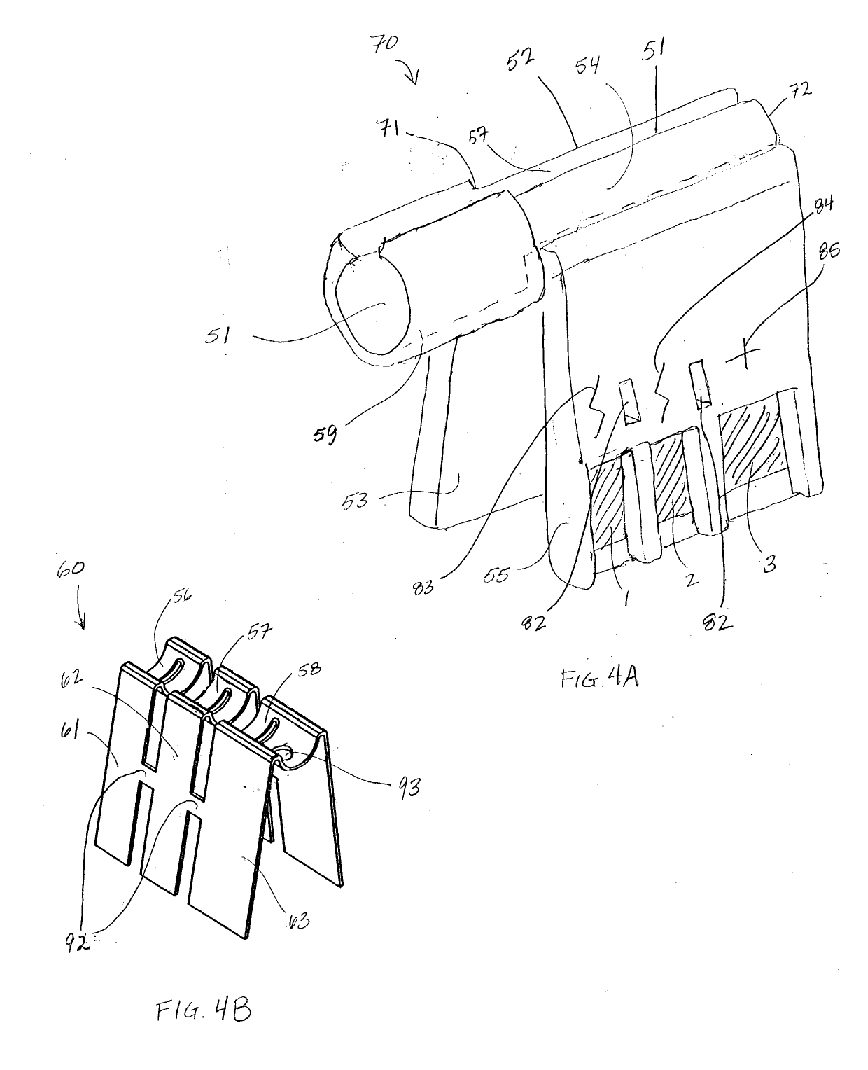

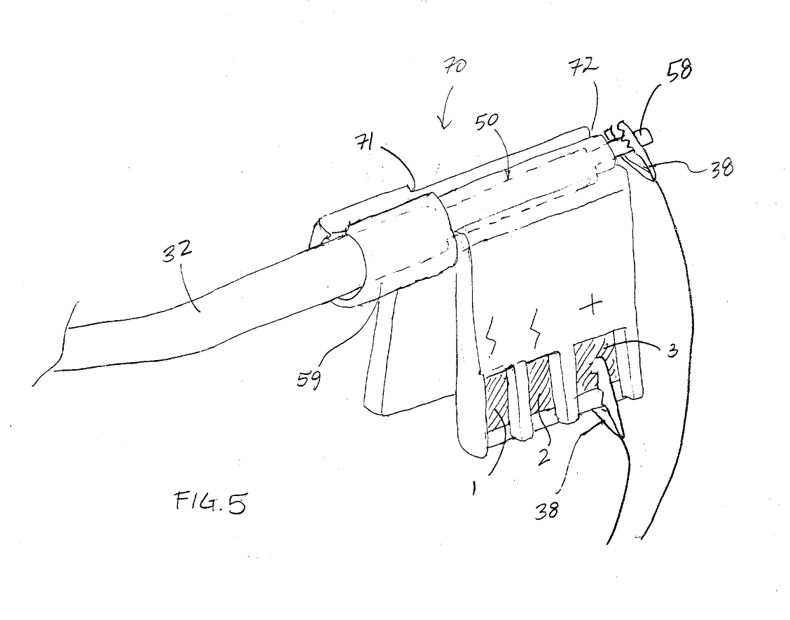

FIG. 4A is an isometric view of an adaptor 70 according to yet another embodiment of the present invention, and FIG. 4B is an isometric view of a conductive assembly 60 included in adaptor 70. FIG. 4B illustrates adaptor 70 including a first housing portion 52 and a second housing portion 54 flexibly joined along a base (not shown) to form a connector receptacle 51 extending longitudinally from a proximal end 72 to a distal end 71 and including a slot 57 likewise extending. A longitudinal extension 59 is shown extending from distal end 71; according to one embodiment of the present invention extension 59 provides strain relief for a lead body of a connector engaged within receptacle 51 (FIG.5), for example lead body 32 of connector 50 illustrated in FIG. 2. It should be noted that extension 59 is not a necessary element in other embodiments of the present invention since a lead may be constructed such that strain relief is not necessary. FIG. 4A further illustrates adaptor 70 including a first lever arm 53 and a second lever arm 55 extending laterally from first housing portion 52 and second housing portion 54; according to embodiments of the present invention an inner surface of receptacle 51 wherein electrical contacts 56, 57, 58 (FIG. 4B) are formed, is dimensioned to tightly engage a lead connector, for example connector 50 shown in FIG. 2; therefore, lever arms 53, 55 are adapted to spread housing portion 52, 54 apart, and thereby open inner surface to allow connector insertion with little to no insertion force required, when lever arms 53, 55 are forced toward one another.

FIG. 4B illustrates conductive assembly 60 including electrical contacts 56, 57, and 58 extending to conductive pathways 61, 62, and 63 respectively. According to embodiments of the present invention, housing portions 52, 54 and lever arms 53, 55 are formed from a relatively hard plastic, which is injection molded over conductive assembly 60 leaving electrical contacts 56, 57, 58 exposed within connector receptacle 51 and portions of conductive pathways 61, 62, and 63 exposed as conductive surfaces 1, 2, and 3, respectively on one or both lever arms 55, 53. A plastic selected for adaptor 70 preferably has physical properties to form a living hinge at base (not shown but corresponding to base 28 shown in FIG. 3A), examples of which include but are not limited to polysulfone, polypropylene and styrene-butadiene. Furthermore, a selected plastic is preferably translucent and may be tinted so that adaptor 70 may be easily seen in a surgical field. FIG. 4B further illustrates a locating feature 93 used to properly orient conductive assembly within a mold for injection molding. After adaptor 70 is molded extensions 92 joining conductive pathways 61, 62, and 63 are punched out, leaving holes 82, in order to physically separate electrical contacts 56, 57, 58 and their associated conductive pathways 61, 62, 63 from one another for electrical isolation. As in previously described embodiments, electrical contacts 56, 57, 58 are adapted to electrically engage contact rings of a connector, for example contact rings 52, 54 and 56 of connector 50 illustrated in FIG. 2, inserted within connector receptacle 51, and are made of any material having an appropriate electrical conductivity and resiliency to allow opening of connector receptacle 51 when lever arms 53, 55 are forced toward one another; examples of appropriate materials include but are not limited to stainless steel, titanium and platinum-iridium alloys.

FIG. 4A further illustrates labels 83, 84, and 85 formed on lever arm 55 corresponding to and identifying each electrical contact, 56, 57, and 58 respectively. Although FIG. 4A shows labels 83 and 84 as symbols representing high voltage and label 85 as a symbol representing a positive polarity or an anode, these symbols may be take on any form to identify electrical contacts according to any application; for example each electrical contact may correspond with a low voltage contact of either polarity. Furthermore labels 83, 84, and 85 may be cut out through lever arm 55, or molded, printed or pasted upon lever arm 55.

FIG. 5 is an isometric view of a lead connector engaged within adaptor 70. As illustrated in FIG. 5, connector 50, shown by partially by dashed lines, is fully engaged within connector receptacle 51 so that pin contact 58 protrudes from proximal end 72 of adaptor 70. FIG. 5 further illustrates a portion of lead body 32 in proximity to connector 50 being strain relieved via engagement within extension 59. According to embodiments of the present invention, lever arms 53 and 55 are forced toward one another to allow easy insertion of connector 50 into receptacle 51 from distal end 71 to proximal end; once connector 50 is fully inserted, arms 53 and 55 are released to allow tight engagement of lead by receptacle 51 wherein electrical contacts 56, 57, and 58 (FIG. 4B) electrically engage ring contacts 52, 54, and 56 (FIG. 2), respectively, and then contact elements, for example alligator clips 38, of an external medical device may be electrically coupled to one or more external contact surfaces 1, 2, 3 and pin contact 58 for electrical testing. It should be noted that, according to one embodiment, extension 59 is adapted to open upon activation of lever arms 53, 55 and may therefore provide a tight fit about lead body 32; in an alternate embodiment extension 59 loosely engages lead body 32 and may or may not be opened up by activation of lever arms 53 and 55 for insertion of connector 50.

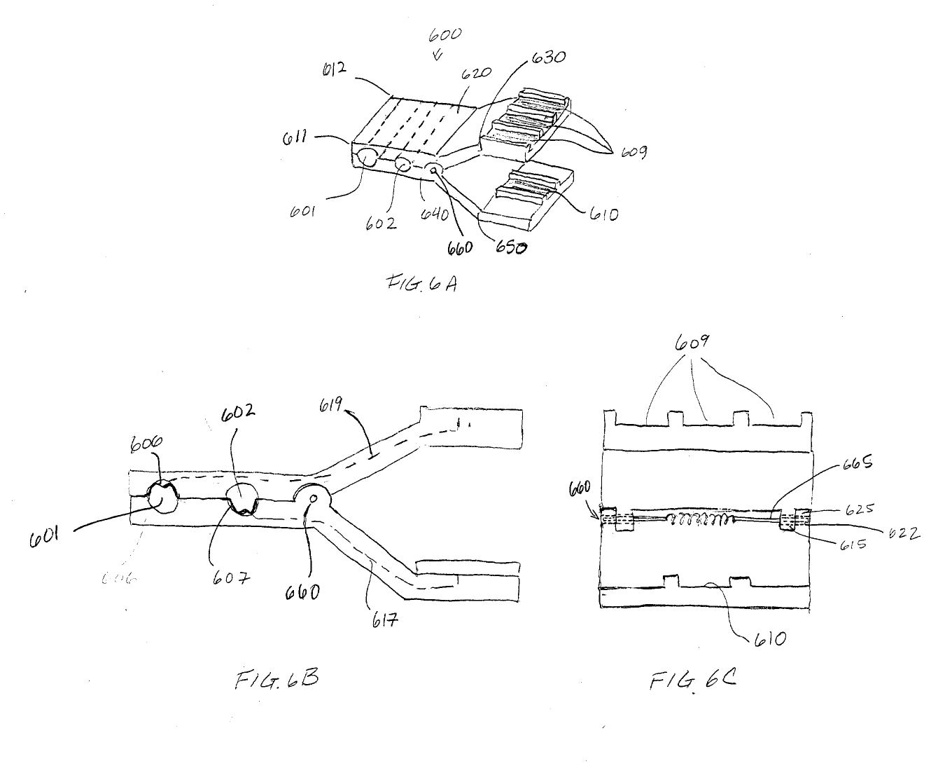

FIG. 6A is an isometric view of an adaptor 600 according to yet another embodiment of the present invention; and FIGS. 6B and 6C are side and end views, respectively, of adaptor 600. As illustrated in FIG. 6A, adaptor 600 includes a first housing portion 620 and second housing portion 640 flexibly joined by a spring hinge assembly 660 and forming a first connector receptacle 601 and second connector receptacle 602, each connector receptacle 601, 602 extending longitudinally from a proximal end 612 to a distal end 611. A first lever arm 630 and a second lever arm 650 are illustrated extending laterally from first housing portion 620 and second housing portion 640; according to embodiments of the present invention lever arms 630, 650 are adapted to spread housing portion 620, 640 apart when lever arms 630, 650 are forced toward one another according to arrows B illustrated in FIG. 6B. According to embodiments of the present invention housing portions 620, 640, and lever arms 630, 650 are formed from a relatively hard plastic, examples of which include but are not limited to polypropylene, polysulfone and styrene-butadiene.

FIG. 6B illustrates first electrical contact 606 formed within first receptacle 601 and electrically coupled to an external contact surface 609 (FIG. 6A) via an internal conductive pathway 619, shown by dashed lines. FIG. 6B further illustrates a second electrical contact 607 formed within second receptacle 602 and electrically coupled to an external contact surface 610 (FIG. 6A) via an internal conductive pathway 617. FIG. 6A shows three external conductive surfaces 609 formed on first lever arm 630, each of which conductive surfaces 609 is electrically coupled, via three conductive pathways 619, to each of three electrical contacts 606 (FIG. 6B) formed within first receptacle 601 and isolated and spaced apart from one another. FIG. 6A further illustrates a single external conductive surface 610 formed on second lever arm 650, which conductive surface 610 is electrically coupled, via conductive pathway 617, to electrical contact 607 (FIG. 6B) formed within second receptacle 602. According to one embodiment of the present invention, inner surface of first receptacle 601 is dimensioned to mate with a first type of connector generally corresponding to connector 50 illustrated in FIG. 2 while an inner surface of second receptacle 602 is dimensioned to mate with a different type of connector generally corresponding to an industry standard IS-1 connector. For each receptacle 601, 601 positions of contacts 606, 607 correspond to positions of ring contacts of corresponding connectors when each connector is fully engaged within receptacles 601, 602; for example, positioning of contacts 606 correspond to positioning of ring contacts 52, 54, 56 of connector 50. In alternate embodiments of the present invention, receptacles 601, 602 may have different numbers of contacts and corresponding conductive pathways 619, 617 and external conductive surfaces 609, 610; furthermore receptacles 601, 602 may have similar dimensions to accommodate similar connectors. According to embodiments of the present invention, electrical contact(s) 606 is coupled to a portion of inner surface of receptacle 601 formed by first housing portion 620, while contact(s) 607 is coupled to a portion of inner surface of receptacle 602 formed by second housing portion 640, and each contact 606, 607 may be made of any conductive material suitable for electrical contacts, examples of which include but are not limited to stainless steel, platinum-iridium alloys, titanium, and gold. As illustrated in FIG. 6B, electrical contacts 606, 607 protrude into receptacles 601, 602, respectively, in order to tightly engage connectors inserted therein for stable electrical connection.

FIG. 6C illustrates spring hinge assembly 660 including a pair of interfacing extensions 615 and 625 from first housing portion 620 and second housing portion 640, respectively, coupled by spring 655 engaged within holes 622 of extensions 615, 625 and extending from proximal end 612 to distal end 611. According to embodiments of the present invention spring hinge assembly 660 flexibly joins first housing portion 620 to second housing portion 640 so that receptacles 601, 602 may tightly engage connectors inserted therein and such that lever arms 630, 650 may be forced together spreading first housing portion 620 apart from second housing portion 640 to open inner surfaces of first receptacle 601 and second receptacle 602 so that connectors may be inserted therein with minimal to no insertion force required. Alternate embodiments of the present invention may utilize different types of spring hinge assemblies, for example one employing a leaf spring rather than a coil spring illustrated in FIG. 6C.

Thus, there has been provided a number of embodiments of medical lead adaptor assemblies, each of which provides a means for electrical coupling between a connector assembly of a cardiac or similar lead with an external medical device wherein sealing zones of the lead connector assembly are protected from contact with conductive probes, clips and the like associated with the external medical device. The inventive medical lead adaptor assembly may be configured to accept lead connectors that may or may not utilize a stylet wire or a guide wire. Furthermore, the inventive lead adaptor may be utilized in conjunction with lead and wires that have compatible lead connector element dimensions; i.e. compatible assemblies in accordance with the present invention may be provided with different dimensions so as to accommodate a variety of cardiac or other types of leads.

While specific embodiments have been presented in the foregoing detailed description of the invention, it should be clear that a vast number of variations exist. It should also be appreciated that the exemplary embodiments are only examples, and are not intended to limit the scope, applicability, or configuration of the invention in any way. Rather, the foregoing detailed description will provide those skilled in the art with a convenient road-map for implementing an exemplary embodiment of the invention. It should be understood that various changes may be made in the function and arrangement of elements described in an exemplary embodiments without departing from the scope of the invention as set forth in the appended claims.

1. An adaptor facilitating an electrical connection between a connector of an implantable medical lead and an external medical device comprising:
a first housing portion and a second housing portion;
a means for flexibly joining the first housing portion to the second housing portion;
a longitudinally extending connector receptacle formed by the first housing portion and the second housing portion, the connector receptacle including a proximal opening, a distal opening and an inner surface dimensioned to tightly engage the connector;
an electrical contact formed within the inner surface of the connector receptacle, positioned in a location corresponding with at least one ring contact of the connector when the connector is fully inserted within the connector receptacle, and adapted to electrically engage the at least one ring contact;
a first lever arm extending laterally from the first housing portion and a second lever arm extending laterally from the second housing portion, the first lever arm and the second lever arm adapted to spread the first housing portion apart from the second housing portion, via the flexible joining means, in order to open the inner surface of the connector receptacle for insertion of the connector into the connector receptacle via the distal opening;
an external contact surface adapted for coupling with a contact element of the external medical device and formed upon the first lever arm; and a conductive pathway electrically coupling the contact surface to the electrical contact;
wherein the connector is fully inserted in the connector receptacle when a proximal pin of the connector protrudes from the proximal end of the connector receptacle.

2. The adaptor of claim 1, wherein the connector receptacle further includes a slot extending from the proximal end of the connector receptacle to the distal end of the connector receptacle.

3. The adaptor of claim 1, wherein the flexible joining means comprises a living hinge integrally formed by a confluence of the first housing portion and the second housing portion.

4. The adaptor of claim 1, wherein the flexible joining means comprises a spring hinge assembly.

5. The adaptor of claim 1, wherein
the electrical contact is formed from a conductive spring material in a generally arcuate shape conforming to the inner surface of the connector receptacle and extending between the first housing portion and the second housing portion; and
the flexible joining means comprises the electrical contact.

6. The adaptor of claim 1, further comprising a longitudinal extension extending from the distal opening of the connector receptacle adapted to provide strain relief for the lead when the connector is fully inserted within the connector receptacle.

7. An adaptor facilitating an electrical connection between a connector of an implantable medical lead and an external medical device comprising:
a first housing portion and a second housing portion;
a means for flexibly joining the first housing portion to the second housing portion;
a longitudinally extending connector receptacle formed by the first housing portion and the second housing portion, the connector receptacle including a proximal opening, a distal opening and an inner surface dimensioned to tightly engage the connector;
a plurality of electrical contacts formed within the inner surface of the connector receptacle, isolated from one another, each positioned in a location corresponding with each of a plurality of ring contacts of the connector when the connector is fully inserted within the connector receptacle and each adapted to electrically engage each of the plurality of ring contacts;
a first lever arm extending laterally from the first housing portion and a second lever arm extending laterally from the second housing portion, the first lever arm and the second lever arm adapted to spread the first housing portion apart from the second housing portion, via the flexible joining means, in order to open the inner surface of the connector receptacle for insertion of the connector into the connector receptacle via the distal opening;
a plurality of external contact surfaces adapted for coupling with one or more contact elements of the external medical device, isolated from one another and formed upon the first lever arm; and
a plurality of conductive pathways isolated from one another and electrically coupling each of the plurality of contact surfaces to each of the plurality of electrical contacts;
wherein the connector is fully inserted in the connector receptacle when a proximal pin of the connector protrudes from the proximal end of the connector receptacle.

8. The adaptor of claim 7, wherein the plurality of electrical contacts includes three electrical contacts, the plurality of external contact surfaces includes three external contact surfaces and the plurality of conductive pathways includes three conductive pathways.

9. The adaptor of claim 8, wherein the connector receptacle further includes a slot extending from the proximal end of the connector receptacle to the distal end of the connector receptacle.

10. The adaptor of claim 8, wherein the flexible joining means comprises a living hinge integrally formed by a confluence of the first housing portion and the second housing portion.

11. The adaptor of claim 8, wherein the flexible joining means comprises a spring hinge assembly.

12. The adaptor of claim 8, wherein
each of the plurality of electrical contacts is formed from a conductive spring material in a generally arcuate shape conforming to the inner surface of the connector receptacle and extending between the first housing portion and the second housing portion; and
the flexible joining means comprises the plurality of electrical contacts.

13. The adaptor of claim 8, further comprising three labels formed on the first lever arm, each of the three labels positioned adjacent to each of the three external contact surfaces for identification of each of the external contact surfaces.

14. The adaptor of claim 8, further comprising a longitudinal extension extending from the distal opening of the connector receptacle adapted to provide strain relief for the lead when the connector is fully inserted within the connector receptacle.

15. An adaptor facilitating an electrical connection between one or more connectors of implantable medical leads and an external medical device comprising:
a first housing portion and a second housing portion;
a means for flexibly joining the first housing portion to the second housing portion;
a first longitudinally extending connector receptacle formed by the first housing portion and the second housing portion, the first connector receptacle including a proximal opening, a distal opening, and an inner surface dimensioned to tightly engage a first connector of the one or more connectors;
a second longitudinally extending connector receptacle formed by the first housing portion and the second housing portion, the second connector receptacle including a proximal opening, a distal opening, and an inner surface dimensioned to tightly engage a second connector of the one or more connectors;
a first lever arm extending laterally from the first housing portion and a second lever arm extending laterally from the second housing portion, the first lever arm and the second lever arm adapted to spread the first housing portion apart from the second housing portion, via the flexible joining means, in order to open the inner surface of the first connector receptacle for insertion of the first connector into the first connector receptacle via the distal opening of the first connector receptacle, and to open the inner surface of the second connector receptacle for insertion of the second connector into the second connector receptacle via the distal opening of the second connector receptacle;
a first electrical contact formed within the inner surface of the first connector receptacle, positioned in a location corresponding with at least one ring contact of the first connector when the first connector is fully inserted within the first connector receptacle, and adapted to electrically engage the at least one ring contact of the first connector;
a second electrical contact formed within the inner surface of the second connector receptacle, positioned in a location corresponding with at least one ring contact of the second connector when the second connector is fully inserted within the connector receptacle, and adapted to electrically engage the at least one ring contact of the second connector; and
a first external contact surface adapted for coupling with a contact element of the external medical device and formed upon the first lever arm;
a second external contact surface adapted for coupling with the contact element of the external medical device and formed upon the second lever arm;
a first conductive pathway electrically coupling the first external contact surface to the first electrical contact; and
a second conductive pathway electrically coupling the second external contact surface to the second electrical contact;
wherein the first connector and the second connector are fully inserted in the first connector receptacle and the second connector receptacle, respectively, when a proximal pin of each of the first connector and the second connector protrudes from the corresponding proximal end of each the first connector receptacle and the second connector receptacle.

16. A method for making electrical connection between a connector of an implantable medical lead and an external medical device, comprising:
spreading a first housing portion apart from a second housing portion via a flexible joining means by forcing toward one another a first lever arm and a second lever arm, the first lever arm extending laterally from the first housing portion and the second lever arm extending laterally from the second housing portion, to open an inner surface of a longitudinally extending connector receptacle formed by the first housing portion and the second housing portion;
inserting the connector into a distal opening of the longitudinally extending connector receptacle until a ring contact of the connector is positioned for electrical engagement by an electrical contact formed within the inner surface of the connector receptacle and a proximal pin contact of the connector protrudes from a proximal end of the connector receptacle; and
coupling a contact element of the external medical device to an external contact surface formed on the first lever arm and coupled to the electrical contact via a conductive pathway.
